(19) United States
(12) Patent Application Publication
(10) Pub. No.: US 2013/0197090 A1
(43) Pub. Date: Aug. 1, 2013

(54) POSTURAL STABILITY AND INCIDENT FUNCTIONS IN PATIENTS

(71) Applicant: Chelsea Therapeutics, Inc., Charlotte, NC (US)

(72) Inventors: L. Arthur Hewitt, Charlotte, NC (US); William Schwieterman, Mobile, AL (US); Cameron Szakacs, Charlotte, NC (US); Gerry Rowse, Waxhaw, NC (US); Christopher Cioffi, Charlotte, NC (US); Michael J. Roberts, Charlotte, NC (US)

(73) Assignee: Chelsea Therapeutics, Inc., Charlotte, NC (US)

(21) Appl. No.: 13/754,001

(22) Filed: Jan. 30, 2013

Related U.S. Application Data
(60) Provisional application No. 61/592,755, filed on Jan. 31, 2012.

(51) Int. Cl. A61K 31/198; A61K 45/06

(52) U.S. Cl. CPC A61K 31/198; A61K 45/06
USPC 514/567

(57) ABSTRACT

The present invention provides methods and systems for reducing falls in patients that are recurrent fallers. Specifically, the compositions, systems, and methods can relate to Parkinson's disease patients, particularly such patients that are suffering from neurogenic orthostatic hypotension. The compositions, systems, and methods comprise the use of droxidopa, optionally in combination with a further active agent. Administration of droxidopa has been found to reduce the mean number of falls per patient per week, as well as provide improvements in the patient's Hoehn and Yahr rating scale score, which is indicative of improvements in postural stability, and provide improvements in the patient's Unified Parkinson's disease Rating Scale score, which is indicative of improvements in the severity of motor and/or non-motor symptoms of Parkinson's disease.

### CROSS-REFERENCE TO RELATED APPLICATIONS

The present application claims priority to U.S. Prov. Pat. App. No. 61/592,755, filed Jan. 31, 2012, the disclosure of which is incorporated herein by reference in its entirety.

### FIELD OF THE DISCLOSURE

The present application is directed to compositions, systems, and methods for improving postural stability in patients. More particularly, the compositions, systems, and methods of the application comprise the administration of droxidopa to the patients. Further, the application is directed to improving functions incident to postural stability, including the ability to stand, remain standing, and resist falls.

### BACKGROUND

Falls are a prominent cause of unintentional injury that can affect many patient populations. In particular, falling can be co-morbid with various chronic illnesses, particularly illnesses that can be characterized by postural instability.

Parkinson's disease (“PD”) is an example of a chronic illness where falls can be a major incapacitating feature of the condition, although the epidemiology of falls in PD is largely unknown within the art. See Bloem et al. (2001) J. Neurol, 248: p. 950-958, which is incorporated herein by reference. Testing has indicated that falls are very common among PD patients, even relatively early in the course of the disease. Estimates show that between 40% and 70% of PD patients fall each year, with one third falling repeatedly. See Balash et al. (2005) J. Neurol., 252: 1310-1315, which is incorporated herein by reference. Moreover, the incidence of falls may be even greater than previously recognized because of underreporting and/or a so-called “amnesia for falls” that may occur in PD patients that are cognitively impaired.

Testing data has indicated elimination of external fall hazards (e.g., stairways, crowded furniture, etc.) may eliminate only a minority of falls in PD patients because such patients tend to suffer from a high proportion of intrinsic falls that are unrelated to environmental hazards. In their testing, Bloem et al. found that patients commonly had center of mass falls (most often while turning around), which suggests that an underlying balance disorder caused most falls. In testing by Ashburn et al. (2008) Disability and Rehabilitation, 30(16): 1205-1212, which is incorporated herein by reference, use of fall diaries by PD patients indicated that 45% of falls occurred when the patient was walking, turning, stepping up or down, or carrying something from one place to another; 32% of falls occurred when the patient was standing, such as when bending toward or reaching for an object, washing, dressing, or completing another everyday task; 21% of falls occurred when a patient was transferring, such as to or from a seat, car, bed, or toilet; and 2% of falls occurred when patients slipped unintentionally out of a chair or rolled out of bed. The testing indicated that the underlying cause for most falls from standing (69%) was loss of balance. On the other hand, freezing, festination, or a leg not moving as expected only accounted for about 5% each of the recorded falls. In still another test by Matinolli et al. (2011) Acta. Neurol. Scand., 123: 193-200, which is incorporated herein by reference, 25% of recurrent fallers in a two year follow up exhibited freezing of gait; however, 51% of the recurrent fallers reported falling unrelated to freezing of gait. Further, 59% of the recurrent fallers suffered from neurogenic orthostatic hypotension (“NOH”), and 47% of non-recurrent fallers suffered from NOH. The testing by Bloem et al. suggested that anti-Parkinson medication did not reduce balance problems since two-thirds of falls in the study occurred when patients considered their symptoms to be well controlled (e.g., with levodopa and/or a dopamine agonist, such as apomorphine). This is consistent with the study by Matinolli et al., wherein 86% of recurrent fallers were taking levodopa as an anti-Parkinson medication. Matinolli et al. concluded that recurrent fallers showed increased postural sway when compared to non-recurrent fallers, and other studies have suggested that postural instability in PD is resistant to conventional pharmacotherapy. See Bloem et al. (1996) Mov. Disord., 11: 509-521; Bonnet et al. (1987) Neurology, 37: 1539-1542; and Klawans, H L (1986) Mov. Disord., 1: 187-192, all of which are incorporated herein by reference.

In particular, in a test by Koller et al. (1989) Clin. Neuropharmacol., 2: 98-105, which is incorporated herein by reference, 38% of PD patients fell, and 13% fell more than once per week. Postural hypotension was uncommon and did not correlate to falling in this study; however, falling did correlate to postural instability, bradykinesia, and rigidity but not with tremor. Frequency of falling in this test was correlated only to the severity of postural instability in PD. Moreover, frequent fallers and postural instability were not changed by dopaminergic therapy, although some fallers with gait difficulties and bradykinesia were improved with levodopa therapy. The conclusion of this study was that frequent falling is caused by postural instability, that such instability is not reversible with dopaminergic therapy (e.g., levodopa), and falling in PD generally does not respond well to drug therapy.

Although levodopa has been used for many years as a therapy for PD patients, some symptoms of PD have been recognized to be non-responsive to levodopa therapy, and this may be ascribed to wide neurodegeneration other than of dopamine neurons. Since levodopa therapy does not provide a significant effect on all symptoms of PD, previous research has been carried out to find alternative therapies. For example, Narabayashi et al. (Proc. Japan Acad., 57, Ser. B, No. 9, 351-354 (1981)) postulated that some symptoms of PD might be due to dysfunction of the norepinephrine nerve system. To activate the norepinephrine nerve system, droxidopa was administered to PD patients suffering from “freezing of gait”, and a beneficial effect was reported. Further studies led to approval of droxidopa for use in Japan (see Narabayashi et al., “clinical evaluation” vol. 15 (No. 3) 423-457 (1987): Clin. Eval., 15: 423-457, 1987, Oct.). However, the efficacy rate of droxidopa in freezing of gait was not necessarily high in the reported studies. In the Societas Neurologica Japonica PA disease guideline, therefore, droxidopa is regarded as a medicament to be optionally tried in PD patients. Nevertheless, the effectiveness of droxidopa in freezing of gait has been previously questioned (see Quinn et al., “Acute administration of DL-threo DOPS does not affect the freezing phenomenon in parkinsonian patients”, Neurology, 1984, 34:149).

Reduction in falls is desirable because of the high rate of adverse consequences including soft tissue injuries, broken bones, and fear of future falls, which can lead to self-imposed restriction of physical activity and even social isolation. Accordingly, there remains a need in the art for further interventions for reducing falls, particularly in PD patients, and more particularly for interventions that are safe and easily introduced to a wide patient population, such as a drug therapy.

### SUMMARY OF THE INVENTION

The present invention provides compositions, systems, and methods for reducing falls in patients, particularly patients with a history of falls and/or patients with an underlying disease or condition that causes a tendency or propensity for falls. More particularly, patients subject to the present invention can include patients subject to characterization of the severity of a falls-related condition. For example, patients that may be treated according to the present invention include Parkinson's disease (“PD”) patients, and the severity of the PD can be characterized by rating scales, such as the Hoehn and Yahr rating scale and versions of the Unified Parkinson's Disease Rating Scale (“UPDRS”).

The compositions, systems, and methods of the invention specifically can comprise administration of droxidopa or a pharmaceutically acceptable ester, amide, salt, solvate, analog, derivative, or prodrug thereof, or a pharmaceutical composition comprising droxidopa or a pharmaceutically acceptable ester, amide, salt, solvate, analog, derivative, or prodrug thereof. The compositions, systems, and methods generally can comprise droxidopa as the sole active agent. In the alternative, the droxidopa can be administered in combination with one or more further pharmaceutically active compounds.

Although the present invention may be described herein in relation to treatment of PD patients, it is understood that the compositions, systems, and methods for reducing falls in patients can extend to any patient or patient population suffering from a disease or condition wherein recurrent falls are a characteristic thereof. Since recurrent falls is a recognized symptom of PD, particularly in PD patients suffering from neurogenic orthostatic hypotension (“NOH”), the present disclosure particularly describes the invention in relation to this condition or combination of conditions. It is intended, however, that the present subject matter encompasses further conditions, as noted above.

In certain embodiments, the invention can provide a method of reducing falls in a PD patient, particularly a PD patient suffering from NOH. Specifically, the method can comprise administering to the patient an effective amount of droxidopa or a pharmaceutically acceptable ester, amide, salt, solvate, analog, derivative, or prodrug thereof. Such reduction of falls may be shown by a post-administration reduction in the mean number of falls per patient per week as compared to a baseline mean number of falls per patient per week before administration of the droxidopa. For example, the post-administration mean number of falls per patient per week may be reduced by at least 20%, by at least 50%, or by a further means of quantification, as otherwise disclosed herein. The number of patient falls may be identified based upon self reporting by the patient, such as via an electronic diary.

In some embodiments, the reduction of falls can be accompanied by a reduction in postural instability. In particular, the reduction in postural instability may be identifiable via a recognized rating scale in the field. For example, the reduction in postural instability may be shown by a post-administration Hoehn and Yahr rating scale score for the patient that is improved as compared to a baseline Hoehn and Yahr rating scale score before administration of the droxidopa. Specifically, the post-administration Hoehn and Yahr rating scale score may be improved by at least 0.2 points, at least 0.3 points, at least 0.4 points, or by a further means of quantification, as otherwise disclosed herein.

As another example, the reduction of falls may be accompanied by a reduction in the severity of PD-related motor and/or non-motor symptoms as measured by the UPDRS scale. Specifically, such reduction in falls may be shown by a UPDRS score for the patient that is improved as compared to a baseline UPDRS score before administration of the droxidopa. More particularly, the post-administration UPDRS score may be improved by at least 4 points, by at least 10 points, or by a further means of quantification, as otherwise disclosed herein.

In further embodiments, the invention can provide a method of improving postural instability in a PD patient. More particularly, such improvement can be in a PD patient exhibiting a baseline Hoehn and Yahr rating scale score that is indicative of postural instability. A minimum score indicative of postural instability, for example, may be a score of at least 0.5, at least 1.0, or another value as otherwise described herein. The method specifically can comprise administering to the patient an effective amount of droxidopa or a pharmaceutically acceptable ester, amide, salt, solvate, analog, derivative, or prodrug thereof. Preferably, the administration is such that a post-administration Hoehn and Yahr rating scale score for the patient is improved as compared to the baseline score. For example, such score may be improved by at least 0.2 points, at least 0.3 points, at least 0.4 points, or by a further means of quantification, as otherwise disclosed herein.

In still other embodiments, the invention can provide a method of improving the severity of motor and/or non-motor symptoms in a PD patient, particularly a PD patient exhibiting a baseline UPDRS score indicative of PD-related motor and/or non-motor symptoms. Such method can comprise administering to the patient an effective amount of droxidopa or a pharmaceutically acceptable ester, amide, salt, solvate, analog, derivative, or prodrug thereof. Preferably, administration is such that a post-administration UPDRS score for the patient is improved as compared to the baseline score. The UPDRS score specifically may be improved by at least 5 points, by at least 10 points, or by a further means of quantification, as otherwise disclosed herein.

In some embodiments, the compositions, systems, and methods of the invention can comprise the use of droxidopa in some combination with one or more additional active agents. Any further active agent recognizable as appropriate for administration to a patient suffering from recurrent falls, such as a PD patient, more particularly a PD patient suffering from NOH, could be combined with droxidopa according to the present invention. In specific embodiments, exemplary active agents for such combination can include DOPA decarboxylase inhibiting compounds, catechol-O-methyltransferase inhibiting compounds, monoamine oxidase inhibiting compounds, cholinesterase inhibiting compounds, and combinations thereof.

When one or more additional active agents are used with the droxidopa according to the present invention, the one or more additional active agents, in some embodiments, can be administered with the droxidopa in a single pharmaceutical composition. In other embodiments, the one or more additional active agents can be administered separately from the droxidopa. The form in which the active agents are administered also can vary according to the invention. For example, in certain embodiments, the droxidopa can be administered in a sustained release form, a controlled release form, or an immediate release form. In other embodiments, the droxidopa specifically may be administered in the form of a mixture enantiomerically enriched in the L-threo isomer. Even further forms for administration are envisioned, as otherwise disclosed herein.

### BRIEF DESCRIPTION OF THE DRAWINGS

Having thus described the invention in general terms, reference will now be made to the accompanying drawings wherein:

FIG. 1 is a graph illustrating the total number of patient falls in the study described in Example 1;

FIG. 2 is a graph illustrating the number of falls per patient per week in the study described in Example 1;

FIG. 3 is a graph illustrating the cumulative distribution of patient falls over time in the study described in Example 1:

FIG. 4 is a graph illustrating the number of patient falls by week reported in the study described in Example 1 as a sensitivity analysis of the full analysis set with the two patients with the highest number of falls excluded from each treatment group;

FIG. 5 is a graph illustrating the number of patient falls by week reported in the study described in Example 1 as a sensitivity analysis of the full analysis set with the five patients with the highest number of falls excluded from each treatment group;

FIG. 6 is a graph illustrating the cumulative distribution of patient falls reported in the study described in Example 1 when the first 10 days of treatment are excluded;

FIG. 7 is a graph illustrating the change from baseline in Hoehn and Fahr rating scale scores at the end of the study described in Example 1;

FIG. 8 is a graph illustrating the change from baseline in MDS-UPDRS scores at the end of the study described in Example 1;

FIG. 9 is a graph illustrating the number of falls per patient per week in the study described in Example 2; and

FIG. 10 is a graph illustrating the number of patient falls by week reported in the study described in Example 2 as a sensitivity analysis of the full analysis set, wherein the number of falls is examined in a group of two patients, a group of five patients, and a group of 10 patients.

### DETAILED DESCRIPTION

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present invention provides compositions, systems, and methods for use in treating a symptom of PD, particularly in treating abnormal postural instability, and more particularly for reducing falls in patients. An occurrence that constitutes a “fall” according to the present invention may follow the World Health Organization definition of “inadvertently coming to rest on the ground, floor or other lower level, excluding intentional change in position to rest in furniture, wall or other objects.” See World Health Organization: WHO Global Report on Falls Prevention in Older Age (2007), which is incorporated herein by reference. In specific embodiments (such as in relation the Example appended hereto), a fall can be defined as an individual unexpectedly coming to rest on the ground, floor, or just a lower level than where the individual started. In further embodiments, a fall alternatively may be defined as any of the following:
“unintentionally coming to the ground or some lower level and other than as a consequence of sustaining violent blow, loss of consciousness, sudden onset of paralysis as in stroke or an epileptic seizure,” (see Gibson et al. (1987) Danish Medical Bulletin 24 (Suppl 4): p. 1-24, which is incorporated herein by reference);
“a fall is a sudden, unintentional change in position causing an individual to land at a lower level, on an object, the floor, of the ground, other than as a consequence of a sudden onset of paralysis, epileptic seizure, or overwhelming external force,” (see Feder et al. (2000) British Medical Journal 321: p. 1007-1011, which is incorporated herein by reference);
“unintentionally coming to rest on the ground, floor, or other lower level” (see Wolf et al. (1996) Journal of the American Geriatrics Society, 44: p. 489-497, which is incorporated herein by reference); or
“an unexpected loss of balance resulting in coming to rest on the floor, the ground or an object below knee level,” (see Lach et al. (1991) Journal of the American Geriatrics Society, 39: p. 197-202, which is incorporated herein by reference).

Any individual that is upwardly mobile is subject to falling. As noted above, however, certain patient populations can be characterized by an increased risk of falls and/or an increased incidence of falls. Such “increased” risk and/or incidence would be understood to be relative to the random occurrences of falls in average adult individuals that do not suffer from any disease or condition that increases risk and/or incidence of falls. Specifically, although such average adult individuals may occasionally fall when performing extraordinary physical activities, falls while carrying out normal, daily activities occur only randomly and/or infrequently. On the contrary, recurrent fallers typically experience an on-going disease or condition that triggers falls with an identifiable frequency. For example, a patient suffering from or exhibiting recurrent falls may be described according to a mean number of falls over a specific time period. Specifically, in certain embodiments, a recurrent faller according to the present invention may be a patient experiencing a mean of at least on fall per month, at least two falls per month, at least three falls per month, at least four falls per month, at least five falls per month, at least six falls per month, at least seven falls per month, or at least eight falls per month. In further embodiments, a recurrent faller according to the present invention may be a patient experiencing a mean of at least 0.1 falls per week, at least 0.2 falls per week, at least 0.4 falls per week, at least 0.5 falls per week, at least 0.6 falls per week, at least 0.8 falls per week, at least 1 fall per week, at least 1.2 falls per week, at least 1.4 falls per week, at least 1.5 falls per week, at least 1.6 falls per week, at least 1.8 falls per week, or at least 2 falls per week. In further embodiments, a recurrent faller according to the present invention may be a patient experiencing a mean of 0.1 to 5 falls per week, 0.1 to 4.5 falls per week, 0.1 to 4 falls per week 0.1 to 3.5 falls per week, 0.1 to 3 falls per week, 0.5 to 5 falls per week, 0.5 to 4.5 falls per week, 0.5 to 4 falls per week 0.5 to 3.5 falls per week, or 0.5 to 3 falls per week. Such mean number of falls preferably is defined by the number of falls suffered by the patient over a period of at least two weeks, at least three weeks, at least four weeks, at least five weeks, at least six weeks, at least two months, at least three months, at least four months, at least five months, at least six months, at least seven months, at least eight months, at least nine months, at least 10 months, at least 11 months, or at least one year.

In some embodiments, the compositions, systems, and methods provided by the present invention thus can apply to a generalized patient population, such as any population of patients that are known to be recurrent fallers or any population of patients that exhibit characteristics of recurrent fallers. In other embodiments, the compositions, systems, and methods of the invention can be applied in a prophylactic manner to patients that have not experienced a fall, but have been diagnosed with a condition that increases the likelihood they may experience a fall. In further embodiments, the compositions, systems, and methods of the invention can apply to patient populations wherein falls can be characterized as being symptomatic of or otherwise relating to a specific underlying cause, such as any population of patients suffering from a group of diseases or conditions for which falls are symptomatic, any population of patients suffering from a specific disease or condition for which falls are symptomatic, or any population of patients exhibiting a physical manifestation of a disease or condition wherein the physical manifestation is related to falls. In certain embodiments, the compositions, systems, and methods can apply to patients exhibiting postural instability. In other embodiments, the compositions, systems, and methods can apply to patients suffering from Parkinson's disease (“PD”). In particular embodiments, the compositions, systems, and methods can apply to PD patients exhibiting abnormal postural instability. In specific embodiments, the compositions, systems, and methods can apply to PD patients suffering from neurogenic orthostatic hypotension (“NOH”). In more specific embodiments, the compositions, systems, and methods can apply to PD patients suffering from NOH and exhibiting postural instability. In further embodiments, the compositions, systems, and methods can apply to patients suffering from PD-related motor symptoms and/or PD-related non-motor symptoms.

In certain embodiments, the compositions, systems, and methods of the invention particularly can be useful for reducing falls specifically in PD patients. As noted previously, falls are a recognized and often incapacitating consequence of PD that is intrinsic to the disease and not typically remedied through elimination of environmental hazards.

The typically recognized cardinal motor symptoms of PD include tremor at rest, rigidity, akinesia (or bradykinesia), and postural instability (as well as flexed posture and freezing). Further PD-related motor symptoms can include hypomimia, dysarthria, dysphagia, sialorrhoea, decreased arm swing, shuffling gait, festination, difficulty arising from a seated position or turning in a lying position, micrographia, difficulty or slowness in carrying out daily living activities (e.g., hygiene, feeding, etc.), glabellar reflex, blepharospasm, dystonia, striatal deformity, scoliosis, and camptocormia.

Although various PD-related motor symptoms may increase a risk of tripping or losing one's footing, it is interesting to note that the testing already described above has indicated that recurrent falls in PD patients is strongly correlative to postural instability. Moreover, such testing generally has indicated that dopaminergic pharmacological interventions have been deemed ineffective in preventing or reducing falls in PD patients, particularly PD patients exhibiting postural instability as a motor symptom of the disease.

Patients suffering from PD also can exhibit non-motor symptoms. Such symptoms can include cognitive impairment, bradyphrenia, tip-of-the-tongue phenomenon, depression, apathy, anhedonia, fatigue, anosmia, ageusia, pain, paresthesia, dysautonomia (including orthostatic hypotension, constipation, urinary and sexual dysfunction, hyperhidrosis, and seborrhea), and sleep disorders (including REM behavior disorder, vivid dreams, daytime drowsiness, sleep fragmentation, and restless leg syndrome).

In embodiments wherein the invention provides for reducing falls in patients, the reduction can be defined by a post-administration reduction in the mean number of falls per patient per unit of time as compared to a baseline mean number of falls per patient per unit of time before treatment according to the invention. For example, a unit of time may be measured in hours, days, weeks, months, or years. Mean number of falls may be characterized in any of the foregoing discussed methods, including any further applicable methods in the field. In specific embodiments, the post-administration mean number of falls per patient per unit of time can be reduced by at least 5%, at least 10%, at least 15%, at least 20%, at least 25%, at least 30%, at least 35%, at least 40%, at least 45%, or at least 50%. In other embodiments, the reduction may be more specifically quantified. For example, the mean number of falls per patient per unit of time may be reduced from at least 3.0 falls to less than 2.5 falls, from at least 3.0 falls to less than 2.0 falls, from at least 3.0 falls to less than 1.5 falls, from at least 3.0 falls to less than 1.0 falls, from at least 3.0 falls to less than 0.5 falls, from at least 2.5 falls to less than 2.0 falls, from at least 2.5 falls to less than 1.5 falls, from at least 2.5 falls to less than 1.0 falls, from at least 2.5 falls to less than 0.5 falls, from at least 2.0 falls to less than 1.5 falls, from at least 2.0 falls to less than 1.0 falls, from at least 2.0 falls to less than 0.5 falls, from at least 1.5 falls to less than 1.0 falls, from at least 1.5 falls to less than 0.5 falls, or from at least 1.0 falls to less than 0.5 falls.

The number of falls suffered by a patient or population of patients can be identified using any useful means in the art, specifically self reporting by the patient—e.g., via an electronic diary. Accordingly, in various embodiments, the invention may be defined in relation to the method by which data for evaluating effectiveness is gathered. For example, the number of falls a patient suffers over a defined time period may be established using self-reporting by the patient or patients.

One method that could be used according to the invention is periodic questionnaires wherein patients (or their informed caregivers) are asked to recall if and how many times the patient has fallen over the defined unit of time. Although this is a useful method, it should be noted that such periodic gathering of data can be prone to under-reporting or over-reporting fall occurrences.

Paper diaries or paper calendars for use in collecting self-reported data on falls can be particularly beneficial because such means relies upon prospective data collection rather than retrospective collection. Thus, paper diaries or calendars recently have been described as the gold standard for self-reported data collection. See Hannan et al. (2010), American Journal of Epidemiology, 171: p. 1031-1036, which is incorporated herein by reference. Such means, however, may be considered somewhat rudimentary in light of technological advances in electronic data collection.

Electronic daily diaries for data collection are believed to be superior to existing paper instruments because of the ability to capture data in “real time”, overcome handwriting difficulties often encountered by PD patients, reduce risk of lost data, and allow for increased integrity through reliable and accurate data. Electronic diaries can include time stamps, reminder functions, and the ability to monitor for compliance as soon as data are entered. Because of these and other advantages, patient compliance for timely completion of study procedures has been observed to be approximately 90% for electron diaries. See Hufford and Shields (April 2002), Applied Clinical Trials: p. 46-56, http://www.ACTmagazine.com, which is incorporated herein by reference.

A number of rating scales are used for the evaluation of impairment and disability in patients with PD. Two such scales are particularly useful for defining the efficacy of the present invention, particularly because of the recognized relationship between postural instability and falls in PD patients, as well as the effect of other PD-related motor symptoms and PD-related non-motor symptoms on falls in PD patients.

The Hoehn and Yahr staging scale is based on the two-fold concept that the severity of overall parkinsonian dysfunction relates to bilateral motor involvement and compromised balance/gait. See Goetz et al. (2004) Movement Disorder Society, 19(9): p. 1020-1028, which is incorporated herein by reference. Thus, the Hoehn and Yahr rating scale is heavily weighted towards postural instability as a primary index of disease severity. As a result, improvements in a PD patient's Hoehn and Yahr rating scale score can serve as a highly useful measure of the effect of treatment in relation to problems associated with motor involvement, compromised balance/gait, and particularly postural instability, such as falls. As reported by Goetz et al., studies of objective and quantitative motor impairment tests and assessments of tasks involved in daily living have identified significant correlations between objective motor performance and Hoehn and Yahr rating scale score. In many instances, progression on the Hoehn and Yahr scale can be a determining factor for initiation of dopaminergic treatments—i.e., levodopa treatment; levodopa treatment has been found to prolong latencies to successive stages on the Hoehn and Yahr scale. Interestingly, few treatments have been shown to positively affect the Hoehn and Yahr score in PD patients. Even with drug treatment of PD that otherwise leads to clinically pertinent improvements, Hoehn and Yahr scale scores do not regularly revert to a lower stage. Moreover, some studies of modern dopaminergic therapies find the percentages of patients reaching the higher stages of the Hoehn and Yahr scale over 10 years to be similar to figures from the pre-levodopa era. Thus, it appears that common PD drug therapies have not previously been shown to positively affect the Hoehn and Yahr scale score, and few treatments, in fact, have been shown to provide Hoehn and Yahr scale score reversion. Accordingly, a statistically significant improvement in the Hoehn and Yahr scale score for a PD patient as a result of a specific treatment can be a surprising indicator of efficacy of the treatment beyond what would normally be expected in the art.

Thus, in further embodiments, the invention can encompass methods of improving postural instability in PD patients. Specifically, the PD patients can be characterized as those exhibiting a baseline Hoehn and Yahr rating scale score indicative of postural instability. In such methods, efficacy may be evidenced by a post-administration Hoehn and Yahr rating scale score for the patient that is improved as compared to a baseline score taken prior to treatment according to the invention.

The compositions, systems, and methods of the present invention more particularly can provide improvement in the Hoehn and Yahr rating scale of a patient, particularly a PD patient, more particularly a PD patient exhibiting postural instability, and even more particularly a PD patient having NOH and also exhibiting postural instability. In such embodiments, the invention can be characterized as reducing falls in the specific patient or patient population, reducing incidence of falls in the specific patient or patient population, or improving postural instability in the specific patient or patient population. In other embodiments, the invention can be characterized specifically as improving the Hoehn and Yahr rating scale score of the specific patient or patient population. In some embodiments, the patient or patient population may be characterized as exhibiting a specific baseline Hoehn and Yahr rating scale score, and such score may be further characterized as being indicative of postural instability. Although postural instability is a characterizing feature for categorizing a patient to fall within a specific stage of the Hoehn and Yahr rating scale, data indicates that the Hoehn and Yahr staging categories should not be strictly applied based upon the stage indicator nomenclature. Blaszczyk et al. (“Assessment of postural instability in patients with Parkinson's disease,” published online Jul. 4, 2007, http://www.cmich.edu/chp/Documents/college_of_healthprofessions/Clinic/bridges/Assessment %20of%20postural%20instability%20in%20patients%20with%20parkinsons%20disease.pdf, which is incorporated herein by reference) carried out testing showing that PD patients with Hoehn and Yahr rating scale scores in the 1-3 range exhibited notable postural instability with recurrent falls. Such testing specifically determined that increased mediolateral sway and sway area while standing with eyes closed are characteristic of parkinsonian postural instability (evident in patients with Hoehn and Yahr rating scale scores across the entire tested range) and is correlative of falls unrelated to freezing. Blaszczyk et al. specifically pointed out that their results confirmed that the deterioration of postural stability control is a continuous process that starts with the onset of the disease, yet efficient compensatory mechanisms can obscure the resulting deficits until late stages of the disease when the compounding effects culminate in an increased recurrence of falls. Thus, Hoehn and Yahr rating scale scores less than 3.0 still can be viewed as being indicative of postural instability, and improvements in postural stability evidenced by an improvement in the Hoehn and Yahr rating scale score as an effect of pharmacological intervention according to the invention is not believed to have yet been shown in the art.

Typically, the Hoehn and Yahr rating scale score is valued between 1 and 5 in 0.5 or 1.0 unit increments. Multiple scorings for an individual patient can be averaged to achieve a mean along a continuous 0-5 scale. Likewise, scorings for a population of patients can be averaged to achieve a mean for the population along a continuous 0-5 scale. Thus, the invention can be characterized such that the post-treatment (or post-administration) Hoehn and Yahr rating scale score is improved by at least 0.2 units, at least 0.3 units, at least 0.4 units, at least 0.5 units, at least 0.6 units, at least 0.7 units, at least 0.8 units, at least 0.9 units, or at least 1.0 units. Even greater improvements may be achieved according to the invention. In other embodiments, the invention can be characterized as improving the Hoehn and Yahr rating scale score for a PD patient by whole units—i.e., by at least 0.5 units, at least 1 unit, at least 1.5 units, or at least 2 units. In further embodiments, the invention can be characterized as improving the Hoehn and Yahr rating scale staging for a PD patient from a pre-treatment (or baseline) stage to a post-treatment stage that is lesser in value. Such improvement can be defined as any of the following: the baseline Hoehn and Yahr score is greater than 4.0 and the post-administration Hoehn and Yahr score is less than 4.0; the baseline Hoehn and Yahr score is greater than 3.0 and the post-administration Hoehn and Yahr score is less than 3.0; the baseline Hoehn and Yahr score is greater than 3.0 and the post-administration Hoehn and Yahr score is less than 2.8; the baseline Hoehn and Yahr score is greater than 3.0 and the post-administration Hoehn and Yahr score is less than 2.5; the baseline Hoehn and Yahr score is greater than 3 and the post-administration Hoehn and Yahr score is less than 2.2; the baseline Hoehn and Yahr score is greater than 2.5 and the post-administration Hoehn and Yahr score is less than 2.5; the baseline Hoehn and Yahr score is greater than 2.5 and the post-administration Hoehn and Yahr score is less than 2.3; the baseline Hoehn and Yahr score is greater than 2.5 and the post-administration Hoehn and Yahr score is less than 2.0; the baseline Hoehn and Yahr score is greater than 2.5 and the post-administration Hoehn and Yahr score is less than 1.8; the baseline Hoehn and Yahr score is greater than 2.0 and the post-administration Hoehn and Yahr score is less than 2.0; the baseline Hoehn and Yahr score is greater than 2.0 and the post-administration Hoehn and Yahr score is less than 1.8; the baseline Hoehn and Yahr score is greater than 2.0 and the post-administration Hoehn and Yahr score is less than 1.5; the baseline Hoehn and Yahr score is greater than 2.0 and the post-administration Hoehn and Yahr score is less than 1.2; the baseline Hoehn and Yahr score is greater than 1.8 and the post-administration Hoehn and Yahr score is less than 1.8; the baseline Hoehn and Yahr score is greater than 1.8 and the post-administration Hoehn and Yahr score is less than 1.5; the baseline Hoehn and Yahr score is greater than 1.8 and the post-administration Hoehn and Yahr score is less than 1.2; or the baseline Hoehn and Yahr score is greater than 1.8 and the post-administration Hoehn and Yahr score is less than 1.0. In still other embodiments, such improvement can be defined as any of the following: the baseline Hoehn and Yahr score is at least 4.0 and the post-administration Hoehn and Yahr score is 3.5 or less; the baseline Hoehn and Yahr score is at least 4 and the post-administration Hoehn and Yahr score is 3.0 or less; the baseline Hoehn and Yahr score is at least 3.5 and the post-administration Hoehn and Yahr score is 3.0 or less; the baseline Hoehn and Yahr score is at least 3.5 and the post-administration Hoehn and Yahr score is 2.5 or less; the baseline Hoehn and Yahr score is at least 3.0 and the post-administration Hoehn and Yahr score is 2.5 or less; the baseline Hoehn and Yahr score is at least 3.0 and the post-administration Hoehn and Yahr score is 2.0 or less; the baseline Hoehn and Yahr score is at least 2.5 and the post-administration Hoehn and Yahr score is 2.0 or less; the baseline Hoehn and Yahr score is at least 2.5 and the post-administration Hoehn and Yahr score is 1.5 or less; the baseline Hoehn and Yahr score is at least 2.0 and the post-administration Hoehn and Yahr score is 1.5 or less; the baseline Hoehn and Yahr score is at least 2.0 and the post-administration Hoehn and Yahr score is 1.0 or less; the baseline Hoehn and Yahr score is at least 1.5 and the post-administration Hoehn and Yahr score is 1.0 or less; or the baseline Hoehn and Yahr score is at least 1.0 and the post-administration Hoehn and Yahr score is 0.5 or less.

The Unified Parkinson's Disease Rating Scale (“UPDRS”) is a well established scale for assessing disability and impairment. Studies making use of the UPDRS to track the progression of PD suggest that the course of PD is not linear and that the rate of deterioration is variable and more rapid in the early phase of the disease and in patients with postural instability and gait difficulty. The UPDRS considers PD-related motor symptoms and PD-related non-motor symptoms in its four component structure (i.e., Part I—mentation, behavior, and mood; Part II—activities of daily living; Part III—motor; and Part IV—complications). Of all available clinical scales for the assessment of parkinsonian motor impairment and disability, the UPDRS is one of the most commonly used instruments, and U.S. and European regulatory agencies have relied on the scale for new drug approvals. Significant improvements in total UPDRS scores, in individual subscales (e.g., Parts II and III), and in averages of subscale scores obtained during on and off scores among fluctuators have been documented in comparison to placebo. Moreover, UPDRS improvements have been seen in patients with studies around new PD treatments.

The UPDRS is based on a series of questions wherein answers are scored on a zero up scale. Higher scores are indicative of increased severity. Total score can be considered as well as subscale scores. An initial UPDRS score (or baseline score) can be evaluated against a post-treatment (or post administration) score wherein a score reduction can be indicative of a lessening of the severity of the patient's (or population's) PD-related motor symptoms, PD-related non-motor symptoms, or PD-related motor and non-motor symptoms. Because the UPDRS score may be evaluated as a whole or on a subscale basis, it is possible to evaluate a UPDRS score as being indicative of PD-related motor symptoms, PD-related non-motor symptoms, or PD-related motor and non-motor symptoms. A patient with a UPDRS score of zero (on a subscale or on the test as a whole) would be viewed as not exhibiting any PD-related motor and/or non-motor symptoms (in relation to the subscale or the test as a whole). A score greater than zero can be viewed as being indicative of PD-related motor and/or non-motor symptoms depending upon whether the score is in a subscale or the test as a whole. Higher scores are indicative of greater severity of the PD-related symptoms.

Thus, the compositions, systems, and methods of the invention may be characterized in relation to an improvement of the severity of PD-related motor symptoms, PD-related non-motor symptoms, or PD-related motor and non-motor symptoms by comparing the score of a post-treatment (or post-administration) UPDRS test with the score of a pre-treatment (or baseline) UPDRS test for the same patient. In various embodiments, the severity of PD-related motor symptoms, PD-related non-motor symptoms, or PD-related motor and non-motor symptoms can be defined as being improved when the post-administration UPDRS score for the patient is improved as compared to the baseline score. In certain embodiments, the improvement can be defined based upon a specific reduction in the UPDRS score. Specifically, the score may be reduced by at least 2 points, at least 4 points, at least 5 points, at least 6 points, at least 8 points, at least 10 points, at least 12 points, at least 14 points, at least 15 points, at least 16 points, at least 18 points, or at least 20 points. Such improvements may relate to the overall UPDRS test score, may relate to the Part I score, may relate to the Part II score, may relate to the Part III score, may relate to the Part IV score, or may relate to any combination of two, or three of the Parts. When the improvement relates to Part III or some combination including Part III, the improvement may be characterized specifically as an improvement in the severity of PD-related motor symptoms. When the improvement relates to a Part or combination of Parts that does not include Part III, the improvement may be characterized specifically as an improvement in the severity of PD-related non-motor symptoms. In further embodiments, the improvement can be defined based upon a percentage change between a post-administration UPDRS score and a baseline UPDRS score. Specifically, the score may be reduced by at least 2%, at least 4%, at least 5%, at least 6%, at least 8%, at least 10%, at least 12%, at least 15%, at least 18%, at least 20%, or at least 25%. Moreover, any improvement as discussed above can relate to the score from a single test for a single patient, the mean of multiple scores for a single patient, or the mean of scores for a population of patients. Thus, the invention expressly relates to treatment of an individual patient, as well as treatment of a population of patients, as otherwise described herein.

As noted previously, a significant number of PD patients also are afflicted with NOH. Thus, the present invention specifically can relate to treatment of PD patients suffering from NOH. Although NOH itself can be a trigger for falls because of dizziness immediately upon standing, the reduction in falls achieved according to the present invention should not mistakenly be considered to arise from a mere lessening of such dizziness immediately upon standing. Rather, the compositions, systems, and methods of the present invention are believed to improve postural stability in PD patients, and this link is supported by the appended Examples wherein the Hoehn and Yahr rating scale scores of PD patients with NOH improved upon application of the compositions, systems, and methods of the present invention. Since the Hoehn and Yahr rating scale score is recognized to strongly relate to postural instability in PD patients, it is believed that the reduction in falls seen with patients using the compositions, systems, and methods of the present invention, particularly PD patients, including PD patients with NOH, arises from an improvement in postural stability. Although it is not believed to be a requirement for defining the present invention, in some embodiments, the present invention may be characterized as reducing falls in PD patients suffering from NOH wherein the reduction arises from an improvement in postural stability of the PD patient and is not strictly related to improvements in dizziness or other symptoms of NOH that typically occur only within a short window of time upon rising from a lying or seated position. The improvement in postural stability may be expressly characterized by an improvement in the PD patient's Hoehn and Yahr rating scale score from a baseline score (before treatment according to the invention) and a post-treatment score. In some embodiments, this improvement in postural stability may be expressly characterized by an improvement in the PD patient's UPDRS score from a baseline score (before treatment according to the invention) and a post-treatment score, particularly wherein the improvement is exhibited in Part III of the UPDRS.

Similarly, an improvement in a PD patient's UPDRS score can be characteristic of a general improvement in the patient's PD-related motor symptoms, as well as the patient's PD-related non-motor symptoms. As already discussed above, in various embodiments, the invention that can be described as reducing the severity of PD-related motor symptoms, PD-related non-motor symptoms, or both PD-related motor and non-motor symptoms as evidenced by a specific reduction in the patient's total UPDRS score (or one, two, or three Parts thereof) from a baseline score before treatment according to the invention and a post-treatment score.

The compositions, systems, and methods of the invention particularly can comprise the use of one or more active agents, which can be administered as one or more pharmaceutical compositions, such as comprising the one or more active agents and one or more pharmaceutically acceptable carriers. Specifically, the compositions, systems, and methods of the invention comprise the use of droxidopa as an active agent.

Droxidopa, is also known as threo-3-(3,4-dihydroxyphenyl) serine, threo-β,3-dihydroxy-L-tyrosine, (−)-(2S,3R)-2-amino-3-hydroxy-3-(3,4-dihydroxyphenyl)propionic acid, and threo-dopaserine, as well as the common terms DOPS, threo-DOPS, and L-DOPS. The structure of droxidopa is provided below in Formula (I).

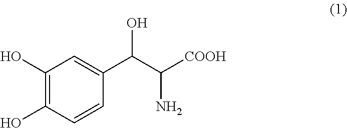 [SMILES: NC(C(=O)O)C(O)c1ccc(O)c(O)c1]

The compound is optically active and can be provided in various forms, including L-threo-DOPS, D-threo-DOPS, L-erythro-DOPS, and D-erythro-DOPS. The compounds can also exist in the racemic form. The L-threo isomer is generally preferred according to the present invention; however, the invention also encompasses compositions and methods of use incorporating the other forms of droxidopa. Accordingly, as used throughout the present disclosure, the term “droxidopa” is intended to encompass any isolated or purified isomer, or isomer enriched mixture (e.g., the L-threo isomer), as well as the racemic forms of droxidopa. When specifically noted, embodiments of the invention expressly can encompass any of the aforementioned isomers and/or racemic forms of droxidopa. For example, the invention specifically can encompass the use of droxidopa that is in the form of a mixture enantiomerically enriched in the L-threo isomer.

Droxidopa is a synthetic amino acid precursor of norepinephrine that is converted directly to norepinephrine via the action of dopa decarboxylase (DDC). Droxidopa has been used to treat neurogenic orthostatic hypotension (NOH) and has been used in treatment of PD patients. Multiple pharmacological activities have been observed with droxidopa, including the following: (1) it is directly converted to 1-norepinephrine by the action of the aromatic L-amino acid decarboxylase which is widely distributed in a living body, and thus has an effect of replenishing norepinephrine; (2) it has limited permeability through the blood-brain barrier into the brain; (3) it specifically recovers norepinephrine activated nerve functions which have decreased in the central and peripheral nervous system; and (4) it shows various actions, as norepinephrine, via the adrenaline receptors in various tissues.

Droxidopa for use according to the invention can be prepared by conventional methods, including methods particularly useful for isolating the L-isomer of droxidopa. See, for example, U.S. Pat. No. 3,920,728; U.S. Pat. No. 4,319,040; U.S. Pat. No. 4,480,109; U.S. Pat. No. 4,562,263; U.S. Pat. No. 4,699,879; U.S. Pat. No. 5,739,387; and U.S. Pat. No. 5,864,041, which are incorporated herein by reference.

The present invention also encompasses pharmaceutically acceptable esters, amides, salts, solvates, and prodrugs of droxidopa. In one embodiment, the invention involves use of droxidopa esters that allow for slowed or delayed decarboxylation of droxidopa resulting from hydrolytic or enzymatic degradation of the ester linkage. As would be recognized by one of skill in the art, an ester of droxidopa can be formed by replacing the hydrogen on the carboxylic ester group with any suitable ester-forming group. For example, U.S. Pat. No. 5,288,898, which is incorporated herein by reference, discloses various esters of N-methylphenylserine, including methyl esters, ethyl esters, n-propyl esters, isopropyl esters, n-butyl esters, isobutyl esters, tert-butyl esters, n-pentyl esters, isopentyl esters, n-hexyl esters, and the like, and the present invention encompasses such esters, as well as other esters. Further examples of ester-forming groups that could be used according to the invention are disclosed in U.S. Pat. No. 5,864,041, which is incorporated herein by reference in its entirety.

In addition to droxidopa, the compositions, systems, and methods of the invention can encompass the use of further active agents. In particular embodiments, an active agent used in combination with droxidopa comprises one or more DOPA decarboxylase (DDC) inhibitors. DDC catalyzes the decarboxylation of levodopa (L-DOPA or 3,4-dihydroxy-L-phenylalanine) and 5-hydroxytryptophan (5-HTP) to yield dopamine and serotonin, respectively. Similarly, DDC catalyzes the conversion of droxidopa to norepinephrine. DDC inhibitors prevent the above-noted conversions and are useful in combination with precursor drugs (such as droxidopa) to focus conversion within the central nervous system and thus increase the concentration of droxidopa in the CNS.

Any compound typically recognized as inhibiting or decreasing the activity of DDC can be used according to the present invention. Non-limiting examples of DDC inhibitors useful according to the invention comprise benserazide, carbidopa, difluoromethyldopa, α-methyldopa, and combinations thereof.

The combination of droxidopa with a DDC inhibitor can be particularly beneficial for focusing the effect of the droxidopa in increasing norepinephrine levels. Many DDC inhibitors, such as benserazide and carbidopa, do not enter the central nervous system. Rather, they remain within the periphery where they prevent decarboxylation of compounds (such as levodopa or droxidopa) into the active metabolites (such as norepinephrine). Thus, when a non-CNS DDC inhibitor is administered in combination with droxidopa, the DDC inhibitor prevents decarboxylation of the droxidopa in the periphery and therefore allows more droxidopa to enter the CNS intact. Once within the CNS (and thus segregated from the DDC inhibitor), the droxidopa can be converted to norepinephrine. Accordingly, the combination of a DDC inhibitor with droxidopa can increase the effective ability of the droxidopa to provide norepinephrine within the CNS and thereby reduce the dose of droxidopa necessary to be effective in treatment.

In further embodiments, an active agent used in combination with droxidopa comprises one or more compounds that at least partially inhibit the function of catechol-O-methyltransferase (such compounds being generally referred to as “COMT inhibitors”). Catechol-O-methyltransferase catalyzes the transfer of the methyl group from S-adenosyl-L-methionine to various catechol compounds (e.g., catecholamines), including dopamine, epinephrine, norepinephrine, and droxidopa. The COMT enzyme is important in the extraneuronal inactivation of catecholamines and drugs with catechol structures, and is generally one of the most important enzymes involved in the metabolism of catecholamines and their metabolites. It is present in most tissues, including the peripheral and the central nervous system.

Inhibitors of COMT slow metabolism and elimination of catechol compounds by increasing their half-life. Accordingly, COMT inhibitors can function to increase levels of naturally occurring catechol compounds, as well as alter the pharmacokinetics of administered catechol compounds (such as L-β-3,4-dihydroxyphenylalanine (L-DOPA), an immediate precursor of dopamine, generally used for symptomatic treatment of Parkinson's disease). Inhibitors of COMT can act peripherally (such as the compound entacapone), while others (such as tolcapone) are capable of crossing the blood-brain barrier and thus acting centrally and peripherally.

Any compound generally recognized as being a COMT inhibitor can be used as an additional active agent according to the invention. Non-limiting examples of COMT inhibitors useful according to the invention include the following: [(E)-2-cyano-N,N-diethyl-3-(3,4-dihydroxy-5-nitrophenyl)propenamide], also called entacapone (COMTAN®); 4-dihydroxy-4′-methyl-5-nitrobenzophenone, also called tolcapone (TASMAR®); and 3-(3,4-dihydroxy-5-nitrophenyl)methylene-2,4-pentanedione, also called nitecapone. In addition to the above examples, U.S. Pat. No. 6,512,136 (the disclosure of which is incorporated herein by reference) describes various substituted 2-phenyl-1-(3,4-dihydroxy-5-nitrophenyl)-1-ethanone compounds that may also be useful as COMT inhibitors according to the present invention. Likewise, U.S. Pat. No. 4,963,590; GB 2 200 109; U.S. Pat. No. 6,150,412; and EP 237 929, each describes groups of COMT inhibiting compounds that could be useful according to the present invention, and the disclosure of each of the above-noted documents is incorporated herein by reference.

Although not wishing to be bound by theory, by providing droxidopa in combination with a COMT inhibitor, it is believed that the ability of the droxidopa to effect treatment is conserved. Specifically, by inhibiting the action of COMT, the COMT inhibiting compound slows or delays the metabolism of droxidopa (as well as norepinephrine itself). This influences the overall plasma concentration of the droxidopa by increasing both the peak plasma concentration (Cₘₐₓ) and the half-life of the administered droxidopa. This is particularly beneficial in that it allows for reduced dosages of droxidopa without limiting effective treatment. Further, the combination of the COMT inhibitor with droxidopa may be effective for increasing the duration of the droxidopa activity (i.e., increasing the duration of norepinephrine activity), which may allow for a reduction in dosing frequency of the droxidopa.

According to another embodiment of the invention, an active agent used in combination with droxidopa comprises one or more compounds that at least partially inhibit the function of cholinesterase. Such cholinesterase inhibiting compounds may also be referred to as anticholinesterase compounds. Cholinesterase inhibiting compounds can be reversible or non-reversible. The present invention preferably encompasses any compounds that may be considered reversible cholinesterase inhibitors (either competitive or non-competitive inhibitors). Non-reversible cholinesterase inhibitors generally find use as pesticides (such as diazinon and Sevin) and chemical weapons (such as tabin and sarin) and are not preferred according to the present invention.

Cholinesterase inhibitors are understood to include compounds that increase levels of acetylcholine (or a cholinergic agonist), generally by reducing or preventing the activity of chemicals involved in the breakdown of acetylcholine, such as acetylcholinesterase. Cholinesterase inhibitors may also include compounds having other mechanisms of action, such as stimulating release of acetylcholine, enhancing response of acetylcholine receptors, or potentiating gonadotropin releasing hormone (GNRH)-induced growth hormone release. Moreover, cholinesterase inhibitors may act by enhancing ganglionic transmission.

Any compound generally recognized as being a cholinesterase inhibitor (or an anticholinesterase compound) may be useful according to the present invention. Non-limiting examples of cholinesterase inhibitors useful in combination with droxidopa according to the invention include the following: 3-dimethylcarbamoyloxy-1-methylpyridinium, also called pyridostigmine (MESTINON® or Regonol); (±)-2,3-dihydro-5,6-dimethoxy-2-[[1-(phenylmethyl)-4-piperidinyl]methyl]-1H-inden-1-one, also called donepezil (ARICEPT); (S)—N-ethyl-3-((1-dimethyl-amino)ethyl)-N-methylphenyl-carbamate, also called rivastigmine (Exelon); (4aS,6R,8aS)-4-a,5,9,10,11,12-hexahydro-3-methoxy-11-methyl-6H-benzofuro [3a, 3, 2ef][2]benzazepin-6-ol, also called galantamine (REMINYL® or RAZADYNE®); 9-amino-1,2,3,4-tetrahydroacridine, also called tacrine (COGNEX®); (m-hydroxyphenyl) trimethylammonium methylsulfate dimethylcarbamate, also called neostigmine; 1-hydroxy-2,2,2-trichloroethylphosphonic acid dimethyl ester, also called metrifonate or trichlorofon; 1,2,3,3A,8,8A-hexahydro-1,3a,8-trimethylpyrrolo-[2,3-b]-indole-5-olmethylcarbamate ester, also called physostigmine; [Oxalylbis(iminoethylene)]-bis-[(o-chlorobenzyl)diethylammonium]dichloride, also called ambenonium (MYTELASE®); ethyl (m-hydroxyphenyl) dimethylammonium, also called edrophonium (ENLON®); demarcarium; thiaphysovenine; phenserine; and cymserine.

More generally, compounds useful as cholinesterase inhibitors according to the invention can comprise carbamate compounds, particularly phenylcarbamates, oganophosphate compounds, piperidines, and phenanthrine derivatives. The invention further comprises cholinesterase inhibitors that are carbamoyl esters, as disclosed in U.S. Published Patent Application No. 2005/0096387, which is incorporated herein by reference.

The above groups of compounds, and specific compounds, are provided to exemplify the types of cholinesterase inhibitors that are useful according to the invention and should not be viewed as limiting the scope of the invention. In fact, the invention can incorporate various further cholinesterase inhibitors, including compounds described in the following documents, the disclosures of which are incorporated herein by reference: Brzostowska, Malgorzata, et al. “Phenylcarbamates of (−)-Eseroline, (−)-N-1-Noreseroline and (−)-Physovenol: Selective Inhibitors of Acetyl and, or Butyrylcholinesterase.” Medical Chemistry Research. (1992) Vol. 2, 238-246; Flippen-Anderson, Judith L., et al. “Thiaphysovenol Phenylcarbamates: X-ray Structures of Biologically Active and Inactive Anticholinesterase Agents.” Heterocycles. (1993) Vol. 36, No. 1; Greig, Nigel H., et al. “Phenserine and Ring C Hetero-Analogues: Drug Candidates for the Treatment of Alzheimer's Disease.” Medicinal Research Reviews. (1995) Vol. 15, No. 1, 3-31; He, Xiao-shu, et al. “Thiaphysovenine and Carbamate Analogues: A New Class of Potent Inhibitors of Cholinesterases.” Medical Chemistry Research. (1992) Vol. 2, 229-237; Lahiri, D. K., et al. “Cholinesterase Inhibitors, β-Amyloid Precursor Protein and Amyloid β-Peptides in Alzheimer's Disease.” Acta Neurologica Scandinavia. (December 2000) Vol. 102 (s176), 60-67; Pei, Xue-Feng, et al. “Total Synthesis of Racemic and Optically Active Compounds Related to Physostigimine and Ring-C Heteroanalogues from 3[-2′-(Dimethylamino0ethyl]-2,3-dihydro-5-methoxy-1,3-dimentyl-1H-indol-2-ol.” Helvetica Chimica ACTA. (1994) Vol. 77; Yu, Qian-sheng, et al. “Total Syntheses and Anticholinesterase Activities of (3aS)—N (8)-Norphysostigmine, (3aS)—N (8)-Norphenserine, Their Antipodal Isomers, and Other N (8)-Substituted Analogues.” J. Med. Chem. (1997) Vol. 40, 2895-2901; and Yu, Q. S., et al. “Novel Phenserine-Based-Selective Inhibitors of Butyrylcholinesterase for Alzheimer's Disease.”Reprinted with permission from J. Med. Chem., May 20, 1999, 42, 1855-1861.

According to yet another embodiment of the invention, an active agent used in combination with droxidopa comprises one or more compounds that at least partially inhibit the function of monoamine oxidase. Monoamine oxidase inhibitors (MAOIs) comprise a class of compounds understood to act by inhibiting the activity of monoamine oxidase, an enzyme generally found in the brain and liver of the human body, which functions to break down monoamine compounds, typically through deamination.

There are two isoforms of monoamine oxidase inhibitors, MAO-A and MAO-B. The MAO-A isoform preferentially deaminates monoamines typically occurring as neurotransmitters (e.g., serotonin, melatonin, epinephrine, norepinephrine, and dopamine). Thus, MAOIs have been historically prescribed as antidepressants and for treatment of other social disorders, such as agoraphobia and social anxiety. The MAO-B isoform preferentially deaminates phenylethylamine and trace amines. Dopamine is equally deaminated by both isoforms. MAOIs may by reversible or non-reversible and may be selective for a specific isoform. For example, the MAOI moclobemide (also known as Manerix or Aurorix) is known to be approximately three times more selective for MAO-A than MAO-B. The invention specifically may encompass MAO-A selective compounds and/or MAO-B selective compounds. Particularly, the MAO-B selective compound rasagiline (AZILECT®) may be used in the invention.

Any compound generally recognized as being an MAOI may be useful according to the present invention. Non-limiting examples of MAOIs useful in combination with droxidopa according to the invention include the following: rasagiline, isocarboxazid (MARPLAN®); moclobemide (Aurorix, Manerix, or Moclodura); phenelzine (NARDIL®); tranylcypromine) (PARNATE°; selegiline (ELDEPRYL®, EMSAM®, or 1-deprenyl); lazabemide; nialamide; iproniazid (marsilid, iprozid, ipronid, rivivol, or propilniazida); iproclozide; toloxatone; harmala; brofaromine (Consonar); benmoxin (Neuralex); and certain tryptamines, such as 5-MeO-DMT (5-Methoxy-N,N-dimethyltryptamine) or 5-MeO-AMT (5-methoxy-α-methyltryptamine).

The combination of droxidopa with an MAOI can provide the effect of conserving bodily norepinephrine levels. In particular embodiments, the MAOI inhibits the action of monoamine oxidase in breaking down norepinephrine, including that formed from the conversion of droxidopa. Accordingly, droxidopa plasma concentrations are positively influenced as the half-life of the droxidopa is increase. This is again particularly beneficial in allowing for reduced droxidopa dosages without limiting effective treatment. Moreover, the combination of the MAOI with droxidopa is also effective for increasing droxidopa activity duration, which again may allow for a reduction in dosing frequency of the droxidopa.

When droxidopa is combined with additional active agents as discussed above, the combination can increase the half-life of droxidopa, and such increase can be seen in a variety of pathways, such as through an effect on drug metabolism, volume of distribution of the drug, or a combination of the two. For example, it has been shown that an increase in droxidopa half-life arising from the combination with a COMT inhibitor, such as entacapone, is indicative of peripheral activity that blocks the metabolism of droxidopa to 3-OM-droxidopa (the major metabolite of droxidopa), thus increasing residence time of droxidopa in the body. Similarly, an increase in the volume of distribution indicates a decrease in the amount of drug available to organs of elimination, which can further affect half-life. Similar effects have been shown when combining droxidopa with MAOIs and cholinesterase inhibitors. Such effects are illustrated in U.S. Pat. Publication No. 2008/0015181, the disclosure of which is incorporated herein by reference in its entirety.

In specific embodiments, in addition to the foregoing compounds, the invention can comprise the use of additional active agents that may be useful in the treatment of PD. Thus, the invention can encompass administration of droxidopa in combination with one or more compounds useful for treating PD or a symptom thereof. In some embodiments, the additional PD treating compound may be a further compound identified as useful for reducing falls. In other embodiments, the additional active agent may be a compound identified as useful for ameliorating a different PD-related symptom or condition.

Biologically active variants of the various compounds disclosed herein as active agents are particularly also encompassed by the invention. Such variants should retain the general biological activity of the original compounds; however, the presence of additional activities would not necessarily limit the use thereof in the present invention. Such activity may be evaluated using standard testing methods and bioassays recognizable by the skilled artisan in the field as generally being useful for identifying such activity.

According to one embodiment of the invention, suitable biologically active variants comprise analogues and derivatives of the compounds described herein. Indeed, a single compound, such as those described herein, may give rise to an entire family of analogues or derivatives having similar activity and, therefore, usefulness according to the present invention. Likewise, a single compound, such as those described herein, may represent a single family member of a greater class of compounds useful according to the present invention. Accordingly, the present invention fully encompasses not only the compounds described herein, but analogues and derivatives of such compounds, particularly those identifiable by methods commonly known in the art and recognizable to the skilled artisan.

The compounds disclosed herein as active agents may contain chiral centers, which may be either of the (R) or (S) configuration, or may comprise a mixture thereof. Accordingly, the present invention also includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the compounds described herein. Various methods are known in the art for preparing optically active forms and determining activity. Such methods include standard tests described herein other similar tests which are will known in the art. Examples of methods that can be used to obtain optical isomers of the compounds according to the present invention are disclosed in U.S. Pat. No. 8,008,285 to Roberts et al., the disclosure of which is incorporated herein by reference in its entirety.

The compound optionally may be provided in a composition that is enantiomerically enriched, such as a mixture of enantiomers in which one enantiomer is present in excess, in particular to the extent of 95% or more, or 98% or more, including 100%.

The compounds described herein as active agents can also be in the form of an ester, amide, salt, solvate, prodrug, or metabolite provided they maintain pharmacological activity according to the present invention. Esters, amides, salts, solvates, prodrugs, and other derivatives of the compounds of the present invention may be prepared according to methods generally known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

Examples of pharmaceutically acceptable salts of the compounds useful according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular example of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxyenzoates, phthalates, sulfonates, xylenesulfonates, phenylacetates, phenylpropionates, phenylbutyrates, citrates, lactates, y-hydroxybutyrates, glycolates, tartrates, methanesulfonates, propanesulfonates, naphthalene-1-sulfonates, naphthalene-2-sulfonates, and mandelates.

An acid addition salt may be reconverted to the free base by treatment with a suitable base. Preparation of basic salts of acid moieties which may be present on a compound useful according to the present invention may be prepared in a similar manner using a pharmaceutically acceptable base, such as sodium hydroxide, potassium hydroxide, ammonium hydroxide, calcium hydroxide, triethylamine, or the like.

Esters of the active agent compounds according to the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C. Examples of pharmaceutically acceptable solvates include, but are not limited to, compounds according to the invention in combination with water, isopropanol, ethanol, methanol, DMSO, ethyl acetate, acetic acid, or ethanolamine.

In the case of solid compositions, it is understood that the compounds used in the methods of the invention may exist in different forms. For example, the compounds may exist in stable and metastable crystalline forms and isotropic and amorphous forms, all of which are intended to be within the scope of the present invention.

If a compound useful as an active agent according to the invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such a p-toluenesulfonic acid or ethanesulfonic acid, or the like.

If a compound described herein as an active agent is an acid, the desired salt may be prepared by any suitable method known to the art, including treatment of the free acid with an inorganic or organic base, such as an amine (primary, secondary or tertiary), an alkali metal or alkaline earth metal hydroxide or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium.

The present invention further includes prodrugs and active metabolites of the active agent compounds described herein. Prodrugs are typically prepared by covalent attachment of a moiety, which results in a compound that is therapeutically inactive until modified by an individual's metabolic system. Any of the compounds described herein can be administered as a prodrug to increase the activity, bioavailability, or stability of the compound or to otherwise alter the properties of the compound. Typical examples of prodrugs include non-active variants of pharmacodynamic compounds that have an art recognized biologically labile protecting group on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound.

A number of prodrug ligands are known. In general, alkylation, acylation, or other lipophilic modification of one or more heteroatoms of the compound, such as a free amine or carboxylic acid residue, reduces polarity and allows passage into cells. Examples of substituent groups that can replace one or more hydrogen atoms on the free amine and/or carboxylic acid moiety include, but are not limited to, the following: aryl; steroids; carbohydrates (including sugars); 1,2-diacylglycerol; alcohols; acyl (including lower acyl); alkyl (including lower alkyl); sulfonate ester (including alkyl or arylalkyl sulfonyl, such as methanesulfonyl and benzyl, wherein the phenyl group is optionally substituted with one or more substituents as provided in the definition of an aryl given herein); optionally substituted arylsulfonyl; lipids (including phospholipids); phosphotidylcholine; phosphocholine; amino acid residues or derivatives; amino acid acyl residues or derivatives; peptides; cholesterols; or other pharmaceutically acceptable leaving groups which, when administered in vivo, provide the free amine and/or carboxylic acid moiety. Any of these can be used in combination with the disclosed active agents to achieve a desired effect.

While it is possible for individual active agent compounds used in the methods of the present invention to be administered in the raw chemical form, it is preferred for the compounds to be delivered as a pharmaceutical composition. Accordingly, there are provided by the present invention pharmaceutical compositions comprising one or more compounds described herein as active agents. As such, the compositions used in the methods of the present invention comprise the pharmaceutically active compounds, as described above, or pharmaceutically acceptable esters, amides, salts, solvates, analogs, derivatives, or prodrugs thereof. Further, the compositions can be prepared and delivered in a variety of combinations. For example, the composition can comprise a single composition containing all of the active agents. Alternately, the composition can comprise multiple compositions comprising separate active agents but intended to be administered simultaneously, in succession, or in another defined period of proximity.

The active agent compounds described herein can be prepared and delivered together with one or more pharmaceutically acceptable carriers therefore, and optionally, other therapeutic agents. Carriers should be acceptable in that they are compatible with any other agents of the composition and not harmful to the recipient thereof. A carrier may also reduce any undesirable side effects of the agent. Such carriers are known in the art. See, Wang et al. (1980) J. Parent. Drug Assn. 34(6):452-462, herein incorporated by reference in its entirety.

Compositions may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety.

Pharmaceutical compositions for use in the methods of the invention are suitable for various modes of delivery, including oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrastemal, and transdermal), topical (including dermal, buccal, and sublingual), vaginal, urethral, and rectal administration. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated.

The pharmaceutical compositions may be conveniently made available in a unit dosage form, whereby such compositions may be prepared by any of the methods generally known in the pharmaceutical arts. Generally speaking, such methods of preparation comprise combining (by various methods) the active compounds of the invention with a suitable carrier or other adjuvant, which may consist of one or more ingredients. The combination of the active agents with the one or more adjuvants is then physically treated to present the composition in a suitable form for delivery (e.g., shaping into a tablet or forming an aqueous suspension).

Pharmaceutical compositions suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions according to the present invention.

In one embodiment, an active agent compound may be administered orally in combination with a pharmaceutically acceptable adjuvant such as an inert diluent or an edible carrier. Oral compositions may be enclosed in hard or soft shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained.

In various embodiments, compositions according to the present disclosure containing the active agent compounds may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present invention may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT®), zein, shellac, and polysaccharides.

Powdered and granular compositions of a pharmaceutical preparation may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of pharmaceutical compositions which are suitable for oral administration may be prepared, packaged, and sold either in liquid form or in the form of a dry product intended for reconstitution with water or another suitable vehicle prior to use.

A tablet containing one or more active agent compounds described herein may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Adjuvants or accessory ingredients for use in the compositions can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluloses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the composition according to the present invention may be anionic, cationic, amphoteric, or nonionic surface active agents. Stabilizers may be included in the compositions to inhibit or lessen reactions leading to decomposition of the active agents, such as oxidative reactions.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Compositions for parenteral administration include aqueous and non-aqueous sterile injection solutions, which may further contain additional agents, such as anti-oxidants, buffers, bacteriostats, and solutes, which render the compositions isotonic with the blood of the intended recipient. The compositions may include aqueous and non-aqueous sterile suspensions, which contain suspending agents and thickening agents. Such compositions for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compositions for use in the methods of the present invention may also be administered transdermally, wherein the active agents are incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agents are contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Compositions for rectal delivery include rectal suppositories, creams, ointments, and liquids. Suppositories may be presented as the active agents in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical compositions may be in any form suitable and readily known in the art for delivery of active agents to the body surface, including dermally, buccally, and sublingually. Typical examples of topical compositions include ointments, creams, gels, pastes, and solutions. Compositions for topical administration in the mouth also include lozenges.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present invention provides medical devices, such as stents, the surface of which is coated with a compound or composition as described herein. The medical device of this invention can be used, for example, in any application for treating, preventing, or otherwise affecting the course of a disease or condition, such as those disclosed herein.

In another embodiment of the invention, pharmaceutical compositions comprising one or more active agents described herein are administered intermittently. Administration of the therapeutically effective dose may be achieved in a continuous manner, as for example with a sustained-release composition, or it may be achieved according to a desired daily dosage regimen, as for example with one, two, three, or more administrations per day. The phrase “time period of discontinuance” is intended to describe a period of discontinuing of the continuous sustained-released or daily administration of the composition. The time period of discontinuance may be longer or shorter than the period of continuous sustained-release or daily administration. During the time period of discontinuance, the level of the components of the composition in the relevant tissue is substantially below the maximum level obtained during the treatment. The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

Administration of the composition comprises administering a pharmaceutically active agent as described herein or administering one or more pharmaceutically active agents described herein in combination with one or more further pharmaceutically active agents (i.e., co-administration). Accordingly, it is recognized that the pharmaceutically active agents described herein can be administered in a fixed combination (i.e., a single pharmaceutical composition that contains both active agents). Alternatively, the pharmaceutically active agents may be administered simultaneously (i.e., separate compositions administered at the same time). In another embodiment, the pharmaceutically active agents are administered sequentially (i.e., administration of one or more pharmaceutically active agents followed by separate administration or one or more pharmaceutically active agents). One of skill in the art will recognize that the desired therapeutic effect will determine the preferred method of administration.

Delivery of a therapeutically effective amount of a composition according to the invention may be obtained via administration of a therapeutically effective dose of the composition. Accordingly, in one embodiment, a therapeutically effective amount is an amount effective to achieve any of the methods of treatment described herein. This includes, but is not limited to: amounts effective to reduce falls in a PD patient, particularly a PD patient suffering from NOH; amounts effective improve postural instability in a PD patient, particularly a PD patient exhibiting a baseline Hoehn and Yahr rating scale score indicative of postural instability; and amounts effective to improve the severity of motor symptoms and/or non-motor symptoms in a PD patient, particularly a PD patient exhibiting a baseline UPDRS score indicative of PD-related motor and/or non-motor symptoms.

The active agents included in the pharmaceutical composition according to the invention are present in an amount sufficient to deliver to a patient a therapeutic amount of an active agent in vivo in the absence of serious toxic effects. The concentration of active agent in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active agent may be administered at once, or may be divided into a number of smaller doses to be administered at varying intervals of time.

A therapeutically effective amount according to the invention can be determined based on the bodyweight of the recipient. Alternatively, a therapeutically effective amount can be described in terms of a fixed dose. In still further embodiments, a therapeutically effective amount of one or more active agents disclosed herein can be described in terms of the peak plasma concentration achieved by administration of the active agents. Of course, it is understood that the therapeutic amount could be divided into a number of fractional dosages administered throughout the day. The effective dosage range of pharmaceutically acceptable salts and prodrugs can be calculated based on the weight and half-life of the parent molecule to be delivered in conjunction with the volume of distribution of the patient. If a salt or prodrug exhibits activity in itself, the effective dosage can be estimated as above using the weight of the salt or prodrug, or by other means known to those skilled in the art.

It is contemplated that compositions of the invention comprising one or more active agents described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

In certain embodiments, a therapeutically effective amount of droxidopa comprises about 10 mg to about 3 g. Such therapeutically effective amount represents an amount of droxidopa that would be provided in a single dose when used as part of a combination according to the invention. It is understood that when the droxidopa is provided as a salt, ester, amide, or other pharmaceutically acceptable form, the amount of the pharmaceutical form of droxidopa can vary to the extent necessary to deliver a therapeutically effective amount of droxidopa. Further, as the therapeutically effective amount of droxidopa is provided as an amount for a single dose, the dosage amounts indicated herein do not necessarily represent the maximum amount of droxidopa that may be administered over the course of a 24 hour period since it is possible that multiple doses of the combination may be indicated for treatment of various conditions.

In further embodiments, the therapeutically effective amount of droxidopa can encompass varying ranges, and the appropriate range could be determined based upon the severity of the condition being treated and the one or more additional compounds with which the droxidopa is combined. In specific embodiments, a therapeutically effective amount of droxidopa comprises about 10 mg to about 2 g, about 10 mg to about 1 g, about 20 mg to about 900 mg, about 30 mg to about 850 mg, about 40 mg to about 800 mg, about 50 mg to about 750 mg, about 60 mg to about 700 mg, about 70 mg to about 650 mg, about 80 mg to about 600 mg, about 90 mg to about 550 mg, about 100 mg to about 500 mg, about 100 mg to about 400 mg, or about 100 mg to about 300 mg.

In yet other embodiments, a therapeutically effective amount of droxidopa can be even greater, such as when provided as a sustained-, extended-, or continuous-release formulation. As understood in the art, such formulations provide an increased drug amount in a single dosage form that slowly releases the drug over time. A therapeutically effective amount of droxidopa for use in such a formulation can be calculated in light of the effective amounts described above and the determined frequency of dosing that would otherwise be necessary to treat a given condition.

A therapeutically effective amount of the one or more additional compounds that are combined with droxidopa according to the invention can be determined in relation to the amount of droxidopa included in the dosage form and the desired ratio of droxidopa to the additional compound(s). Advantageously, the present invention allows for great flexibility in formulating combinations. For example, the conserving effects provided by the one or more additional compounds can allow for using droxidopa in a lesser amount and still achieve the same, or better, therapeutic effects achieved using droxidopa alone. Likewise, it is possible to increase the therapeutic effects of droxidopa by using an amount of the one or more additional compounds that is less than the typically recommended dosage for the one or more additional compounds.

In one embodiment, the ratio of droxidopa to the one or more additional compounds is in the range of about 500:1 to about 1:10. In further embodiments, the ratio of droxidopa to the additional compound(s) is in the range of about 250:1 to about 1:5, about 100:1 to about 1:2, about 80:1 to about 1:1, about 50:1 to about 2:1, or about 20:1 to about 3:1.

The one or more additional compounds combined with droxidopa according to the invention can be included in amount typically recommended for use of the compounds alone for other indications. However, as noted above, it is possible according to the invention to use the additional compound(s) in amounts that are less than typically recommended, particularly in relation to DDC inhibitors, COMT inhibitors, cholinesterase inhibitors, and MAO inhibitors. In certain embodiments, a therapeutically effective amount of a DDC inhibitor, COMT inhibitor, cholinesterase inhibitor, or MAO inhibitor to be combined with droxidopa is in the range of about 1 mg to about 200 mg. Of course, this range is exemplary and could vary depending upon the amount of droxidopa included in the combination and the desired ratio of the compounds in the combination, as described above.

The present invention also includes an article of manufacture providing a composition comprising one or more active agents described herein. The article of manufacture can include a vial or other container that contains a composition suitable for use according to the present invention together with any carrier, either dried or in liquid form. In particular, the article of manufacture can comprise a kit including a container with a composition according to the invention. In such a kit, the composition can be delivered in a variety of different combinations. For example, the composition can comprise a single dosage comprising all of the active agents. Alternately, where more than one active agent is provided, the composition can comprise multiple dosages, each comprising one or more active agents, the dosages being intended for administration in combination, in succession, or otherwise in close proximity of time. For example, the dosages could be solid forms (e.g., tablets, caplets, capsules, or the like) or liquid forms (e.g., vials), each comprising a single active agent, but being provided in blister packs, bags, or the like, for administration in combination.

The article of manufacture further includes instructions in the form of a label on the container and/or in the form of an insert included in a box in which the container is packaged, for the carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

### EXPERIMENTAL

The present invention will now be described with specific reference to the following examples, which are not intended to be limiting of the invention and are rather provided as exemplary embodiments. The examples illustrate the effect of droxidopa for reducing falls in patients with Parkinson's disease.

### Example 1

A multi-center, double-blind, randomized, parallel-group, placebo-controlled study was carried out to assess the clinical effect of droxidopa over the course of 10 weeks for reducing falls in PD patients. The study included a two week double-blind dose-titration period followed by an eight week double-blind treatment period. A screening period (up to 14 days) was used to determine patient eligibility. Throughout the study, visit specific assessments were conducted three hours (an acceptable range was two to five hours) following the patient's first daily dose of droxidopa. Patients who successfully passed all screening assessments continued to the baseline measurements. At the end of the baseline visit, eligible patients were randomized to treatment with either droxidopa or placebo (randomization was double-blind, 1:1). Patients entered a double-blind titration phase at 100 mg TID of droxidopa or matching placebo. Treatment was escalated in 100 mg TID increments to a maximum of 600 mg TID. Upon completion of the dose titration phase, patients returned for a study visit after 8 weeks of double-blind treatment at their titrated dose. The study included 27 placebo-treated patients and 24 droxidopa-treated patients.

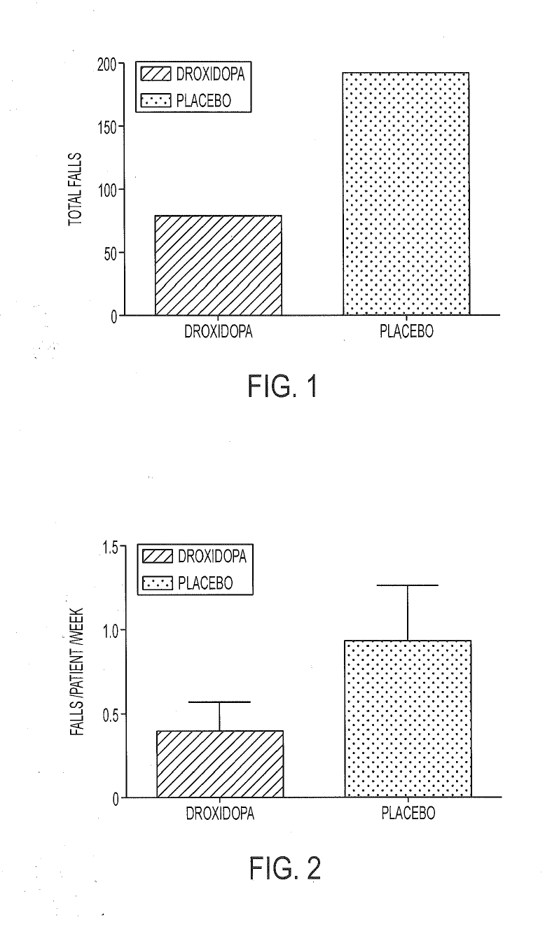

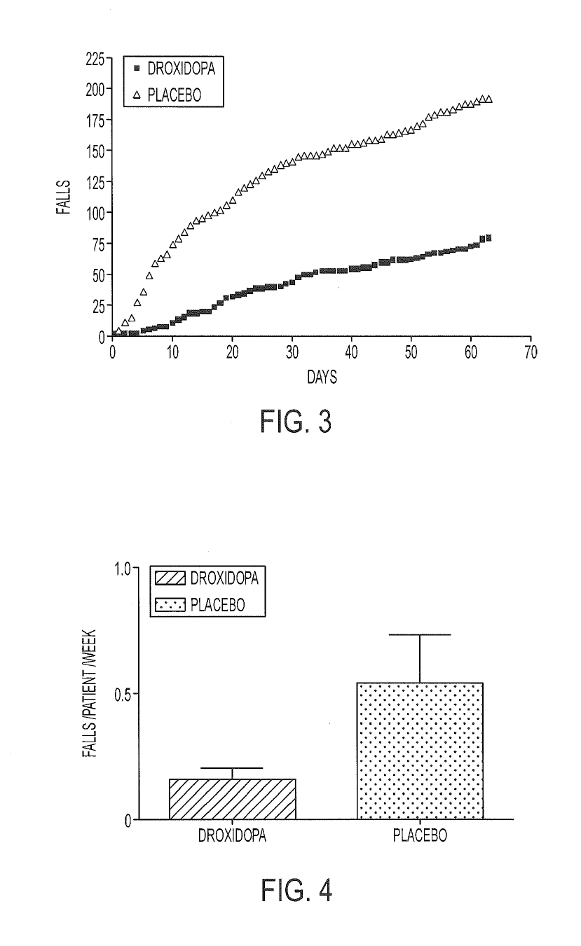

During the study, the placebo-treated patients reported a total of 197 falls compared with 79 falls reported by the droxidopa-treated patients. See FIG. 1. Over one-third of the patients did not experience any falls, while a minority (nine patients receiving droxidopa and 12 patients receiving placebo) experienced recurrent falls. Standardizing the above data to the number of falls per patient per week, patients randomized to the placebo treatment experienced an average of 0.93 falls per patient per week. Patients randomized to the droxidopa treatment experienced an average of 0.39 falls per patient per week. See FIG. 2. This represents an approximate 60% reduction in the number of falls reported by droxidopa-treated patients compared with placebo-treated patients. Based on an analysis of the cumulative number of patient-recorded falls, the relative increase in the number of falls in the placebo treatment group over time was consistently larger compared with the relative increase over time in the droxidopa treatment group. See FIG. 3.

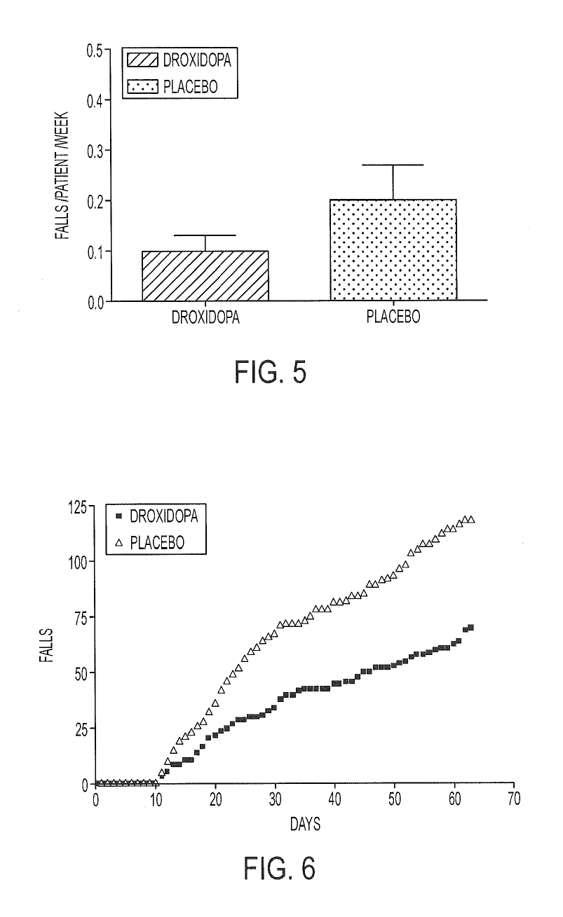

In the study, it was evident that a number of patients in each group was not normally distributed—i.e., a few patients in each group experienced many more falls than the remaining patients in each respective group. A sensitivity analysis was performed to assess the robustness of the treatment effect in light of this disparity in falls within each group. The beneficial treatment effect favoring droxidopa was evident even when the top two patients or the top five patients who experienced the highest number of falls in each treatment group were removed from the analysis. See FIG. 4 and FIG. 5, respectively. When the top two patients in each treatment group were excluded, patients randomized to the placebo group experienced an average of 0.54 falls per patient per week compared with 0.16 falls per patient per week in patients randomized to the droxidopa group. Similarly, when the top five patients in each treatment group were excluded, patients randomized to the placebo group experienced an average of 0.20 falls per patient per week compared with 0.10 falls per patient per week in patients randomized to the droxidopa group. In addition, when data from the first 10 days of the study were removed, the beneficial treatment effect favoring droxidopa on the total number of patient falls remained evident. See FIG. 6.

Patients in the study were evaluated using the Hoehn and Yahr rating scale and the Movement Disorder Society-sponsored revised version of the UPDRS to evaluate the effect of treatment as evidenced by these two scales. A baseline score for each patient using each test was established prior to treatment with droxidopa or the placebo, and a post-treatment score for each test was established at the end of the study. Patients with recurrent falls treated with droxidopa experienced a marked improvement in both the Hoehn and Yahr score and in the UPDRS score than did patients with recurrent falls treated with placebo. This data is summarized below in Table 1a, wherein recurrent fallers are defined as those patients who experienced greater than 1 fall during the course of the study, and non-recurrent fallers are defined as those patients who experienced either zero or one fall during the study.

TABLE 1a

| | Hoehn and Yahr | | MDS-UPDRS | |
|---|---|---|---|---|
| Treatment Group | Baseline | End of Study | Baseline | End of Study |
| Recurrent Fallers | | | | |
| Droxidopa (n = 9) | 1.9 | 1.7 | 89.1 | 77.7 |
| Placebo (n = 12) | 2.3 | 2.6 | 87.6 | 90.0 |
| Non-recurrent | | | | |
| Fallers | | | | |
| Droxidopa (n = 15) | 1.8 | 1.4 | 74.3 | 58.1 |
| Placebo (n = 15) | 2.1 | 2.0 | 91.4 | 71.8 |

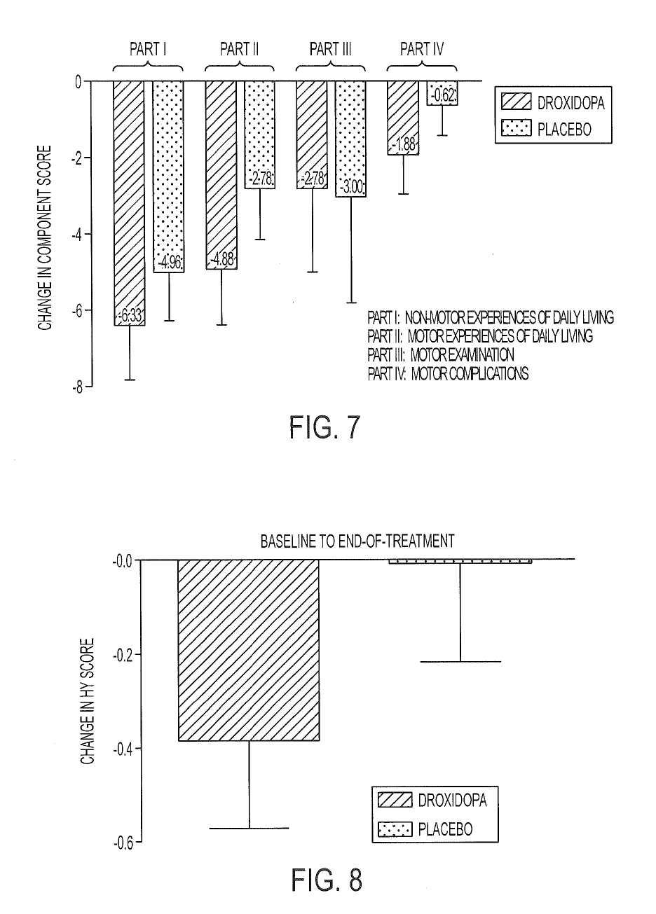

Since the Hoehn and Yahr rating scale is heavily weighted towards postural instability as the primary index of disease severity, it is believed that the Hoehn and Yahr score serves as a useful measure of the effect of treatment on NOH in a PD patient population. At the end of this study, droxidopa-treated patients experienced, on average, an improvement from baseline (i.e., a reduction in the score) in their Hoehn and Yahr rating scale score (change: −0.4 points) while placebo-treated patients experienced no change from baseline in their Hoehn and Yahr rating scale score, resulting in a notable treatment difference of −0.4 units favoring droxidopa. See FIG. 7. Based on an analysis of Hoehn and Yahr rating scale scores in individual patients, a higher proportion of droxidopa-treated patients experienced an improvement in their Hoehn and Yahr rating scale score at the end of the study (10 of 24 patients; 42%) compared with placebo-treated patients (5 of 27 patients; 19%). Conversely, a lower proportion of droxidopa-treated patients experienced a worsening in their Hoehn and Yahr rating scale score at the end of the study (3 of 24 patients; 13%) compared with placebo-treated patients (10 of 27 patients; 37%). This data is summarized below in Table 1b.

TABLE 1b

| Change in Hoehn and | Placebo | Droxidopa |
|---|---|---|
| Yahr Rating Scale Score | (N = 27) | (N = 24) |
| Worsened, n (%) | 10 (37) | 3 (13) |
| No Change n (%) | 12 (44) | 11 (46) |
| Improved, n (%) | 5 (19) | 10 (42) |

The UPDRS likewise is a useful test for evaluating improvements in symptoms that can be a cause of falls in PD patients since this scale is used to assess the severity of motor and non-motor symptoms in PD patients. In the present study, droxidopa-treated patients experienced a 15.8 point improvement (i.e., decrease) from baseline compared with an 11.4 point improvement in placebo-treated patients. See FIG. 8. Additionally, each of the four components of the UPDRS score also showed benefits for droxidopa compared with placebo. The 4.4 point treatment difference favoring droxidopa in the total UPDRS score reflects pronounced improvement in the motor features of PD and mirrors the improvements observed on the Hoehn and Yahr rating scale score.

### Example 2

A multi-center, double-blind, randomized, placebo-controlled study was carried out to assess the clinical effect of droxidopa for reducing falls in PD patients. The study included a two week double-blind dose-titration period followed by an eight week double-blind treatment period. A screening period (up to 14 days) was used to determine patient eligibility. The patients were then randomized into a treatment group and a placebo group. Patients entered a double-blind titration phase at 100 mg TID of droxidopa or matching placebo. Treatment was escalated in 100 mg TID increments to a maximum of 600 mg TID. Upon completion of the dose titration phase, patients returned for study visits after 1, 2, 4, and 8 weeks of double-blind treatment at their titrated dose. The study included 78 placebo-treated patients and 69 droxidopa-treated patients.

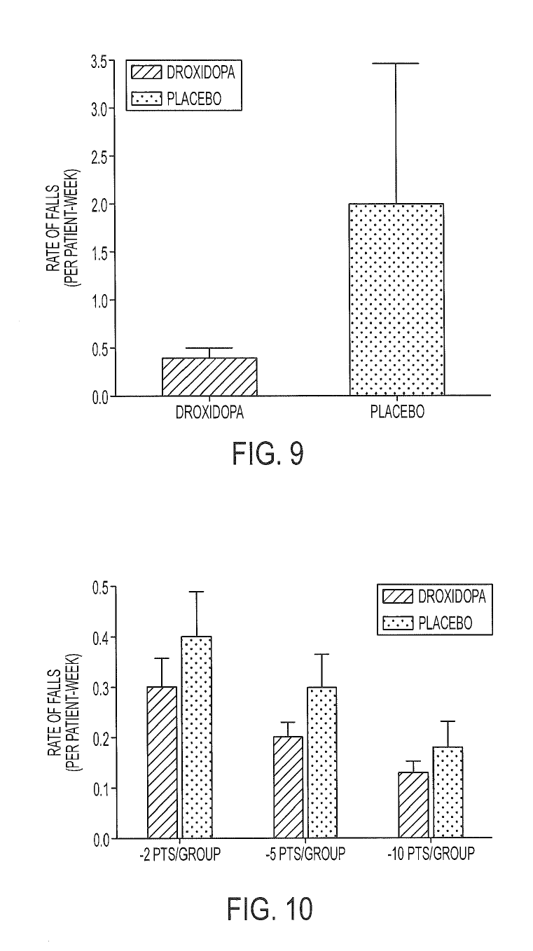

The study results showed that the rate of falls per patient per week for the droxidopa treatment group was visibly less than in the placebo treatment group. This is illustrated in FIG. 9. The beneficial treatment effect favoring droxidopa was again evident even when the top two patients, the top five patients, and the top ten patients who experienced the highest number of falls in each treatment group were removed from the analysis. See FIG. 10 for these results.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method of improving postural instability in a Parkinson's disease (PD) patient, the method comprising administering an effective amount of droxidopa or a pharmaceutically acceptable ester, amide, salt, solvate, or prodrug thereof to the PD patient.

2. The method of claim 1, wherein the improved postural instability is defined by a reduction in falls.

3. The method of claim 2, wherein the reduction of falls is defined by a post-administration reduction in the mean number of falls per patient per week as compared to a baseline mean number of falls per patient per week before administration of the droxidopa or a pharmaceutically acceptable ester, amide, salt, solvate, or prodrug thereof.

4. The method of claim 3, wherein the post-administration mean number of falls per patient per week is reduced by at least 20%.

5. The method of claim 3, wherein the post-administration mean number of falls per patient per week is reduced by at least 50%.

6. The method of claim 2, wherein the number of falls is identified based upon reporting by the patient or the patient's caregiver.

7. The method of claim 2, wherein prior to administration, the PD patient is a recurrent faller.

8. The method of claim 7, wherein the PD patient has experienced a mean of at least 0.2 falls per week over a period of at least six weeks prior to administration.

9. The method of claim 1, wherein the postural instability is defined by a baseline Hoehn and Yahr rating scale score indicative of the postural instability, and wherein the improvement in the postural instability is defined by a post-administration Hoehn and Yahr rating scale score for the patient that is improved as compared to the baseline score.

10. The method of claim 9, wherein the post-administration Hoehn and Yahr rating scale score is improved by at least 0.2 points.

11. The method of claim 9, wherein the post-administration Hoehn and Yahr rating scale score is improved by at least 0.3 points.

12. The method of claim 9, wherein the post-administration Hoehn and Yahr rating scale score is improved by at least 0.4 points.

13. The method of claim 9, wherein the improvement is defined by at least one of the following:
the baseline Hoehn and Yahr score is greater than 4.0 and the post-administration Hoehn and Yahr score is less than 4.0;
the baseline Hoehn and Yahr score is greater than 3.0 and the post-administration Hoehn and Yahr score is less than 3.0;
the baseline Hoehn and Yahr score is greater than 3.0 and the post-administration Hoehn and Yahr score is less than 2.8;
the baseline Hoehn and Yahr score is greater than 3.0 and the post-administration Hoehn and Yahr score is less than 2.5;
the baseline Hoehn and Yahr score is greater than 3.0 and the post-administration Hoehn and Yahr score is less than 2.2;
the baseline Hoehn and Yahr score is greater than 2.5 and the post-administration Hoehn and Yahr score is less than 2.5;
the baseline Hoehn and Yahr score is greater than 2.5 and the post-administration Hoehn and Yahr score is less than 2.3;
the baseline Hoehn and Yahr score is greater than 2.5 and the post-administration Hoehn and Yahr score is less than 2.0;
the baseline Hoehn and Yahr score is greater than 2.5 and the post-administration Hoehn and Yahr score is less than 1.8;
the baseline Hoehn and Yahr score is greater than 2.0 and the post-administration Hoehn and Yahr score is less than 2.0;
the baseline Hoehn and Yahr score is greater than 2.0 and the post-administration Hoehn and Yahr score is less than 1.8;
the baseline Hoehn and Yahr score is greater than 2.0 and the post-administration Hoehn and Yahr score is less than 1.5;
the baseline Hoehn and Yahr score is greater than 2.0 and the post-administration Hoehn and Yahr score is less than 1.2;
the baseline Hoehn and Yahr score is greater than 1.8 and the post-administration Hoehn and Yahr score is less than 1.8;
the baseline Hoehn and Yahr score is greater than 1.8 and the post-administration Hoehn and Yahr score is less than 1.5;
the baseline Hoehn and Yahr score is greater than 1.8 and the post-administration Hoehn and Yahr score is less than 1.2; or
the baseline Hoehn and Yahr score is greater than 1.8 and the post-administration Hoehn and Yahr score is less than 1.0.

14. The method of claim 9, wherein the improvement is defined by at least one of the following:
the baseline Hoehn and Yahr score is at least 4 and the post-administration Hoehn and Yahr score is 3.5 or less;
the baseline Hoehn and Yahr score is at least 4 and the post-administration Hoehn and Yahr score is 3.0 or less;
the baseline Hoehn and Yahr score is at least 3.5 and the post-administration Hoehn and Yahr score is 3.0 or less;
the baseline Hoehn and Yahr score is at least 3.5 and the post-administration Hoehn and Yahr score is 2.5 or less;
the baseline Hoehn and Yahr score is at least 3.0 and the post-administration Hoehn and Yahr score is 2.5 or less;
the baseline Hoehn and Yahr score is at least 3.0 and the post-administration Hoehn and Yahr score is 2.0 or less;
the baseline Hoehn and Yahr score is at least 2.5 and the post-administration Hoehn and Yahr score is 2.0 or less;
the baseline Hoehn and Yahr score is at least 2.5 and the post-administration Hoehn and Yahr score is 1.5 or less;
the baseline Hoehn and Yahr score is at least 2.0 and the post-administration Hoehn and Yahr score is 1.5 or less;
the baseline Hoehn and Yahr score is at least 2.0 and the post-administration Hoehn and Yahr score is 1.0 or less;
the baseline Hoehn and Yahr score is at least 1.5 and the post-administration Hoehn and Yahr score is 1.0 or less; or
the baseline Hoehn and Yahr score is at least 1.0 and the post-administration Hoehn and Yahr score is 0.5 or less.

15. The method of claim 1, wherein the postural instability is defined by a baseline Unified Parkinson's Disease Rating Scale (UPDRS) score indicative of PD-related motor or non-motor symptoms, and wherein the improvement in the postural instability is defined by a post-administration UPDRS score for the patient that is improved as compared to the baseline score.

16. The method of claim 15, wherein the post-administration UPDRS score is improved by at least 5 points.

17. The method of claim 15, wherein the post-administration UPDRS score is improved by at least 10 points.

18. The method of claim 15, wherein the symptom is a motor symptom.

19. The method of claim 18, wherein the motor symptom is related to recurrent falls in the PD patient.

20. The method of claim 1, wherein PD patient further suffers from neurogenic orthostatic hypotension (NOH).

21. The method of claim 1, further comprising administering one or more additional active agents selected from the group consisting of DOPA decarboxylase inhibiting compounds, catechol-O-methyltransferase inhibiting compounds, monoamine oxidase inhibiting compounds, cholinesterase inhibiting compounds, and combinations thereof.

22. The method of claim 21, wherein the one or more additional active agents are administered with the droxidopa or a pharmaceutically acceptable ester, amide, salt, solvate, or prodrug thereof in a single pharmaceutical composition.

23. The method of claim 21, wherein the one or more additional active agents are administered separately from the droxidopa or a pharmaceutically acceptable ester, amide, salt, solvate, or prodrug thereof.

24. The method of claim 1, wherein the droxidopa or a pharmaceutically acceptable ester, amide, salt, solvate, or prodrug thereof is administered in a sustained release form.

25. The method of claim 1, wherein the droxidopa or a pharmaceutically acceptable ester, amide, salt, solvate, or prodrug thereof is administered in a controlled release form.

26. The method of claim 1, wherein the droxidopa or a pharmaceutically acceptable ester, amide, salt, solvate, or prodrug thereof is administered in an immediate release form.

27. The method of claim 1, wherein droxidopa or a pharmaceutically acceptable ester, amide, salt, solvate, or prodrug thereof is administered in the form of a mixture enantiomerically enriched in the L-threo isomer.
